(19) United States
(12) Patent Application Publication
(10) Pub. No.: US 2012/0115804 A1
(43) Pub. Date: May 10, 2012

(54) COMPOSITIONS AND METHODS FOR CHARACTERIZING AND TREATING NEOPLASIA

(75) Inventors: Radharani Marik, Baltimore, MD (US); Christopher B. Umbricht, Baltimore, MD (US); Martha A. Zeiger, Baltimore, MD (US)

(73) Assignee: The John Hopkins University, Baltimore, MD (US)

(21) Appl. No.: 13/255,780

(22) PCT Filed: Mar. 10, 2010
(86) PCT No.: PCT/US2010/000741

§ 371 Date: Jan. 19, 2012

Related U.S. Application Data
(60) Provisional application No. 61/159,038, filed on Mar. 10, 2009.

(51) Int. Cl. C12Q 1/68; C07H 21/04; A61K 31/167; A61P 35/00; A61K 31/4353; A61K 31/165; A61K 31/166; A61K 31/706; A61K 31/593; A61K 31/19

(52) U.S. Cl. 514/43; 435/6.12; 514/167; 536/24.33; 514/575; 514/557; 514/300; 514/620

(57) ABSTRACT

The invention features compositions and methods for characterizing the methylation status of the Vitamin D Receptor (VDR) in neoplasia (e.g., breast carcinoma), selecting an appropriate therapy, and treating the neoplasia (e.g., breast carcinoma).

### CROSS-REFERENCE TO RELATED APPLICATION

This application claims the benefit of the following U.S. Provisional Application No. 61/159,038, filed Mar. 10, 2009, the entire contents of which are incorporated herein by reference.

### STATEMENT OF RIGHTS TO INVENTIONS MADE UNDER FEDERALLY SPONSORED RESEARCH

This work was supported by the following grants from the National Institutes of Health, Grant Nos: NCI P50 CA088843-06A1 and the Department of Defense DAMD17-03-1-0547 (CBU). The government has certain rights in the invention.

### BACKGROUND OF THE INVENTION

Each year, approximately 200,000 women in the United States are diagnosed with breast cancer, and one in nine American women will develop breast cancer in her lifetime. Although methods for treating breast cancer have improved in recent years, breast cancer kills more women in the United States than any cancer except lung cancer. Early detection and treatment of breast cancer improves the odds that women diagnosed with this devastating disease will survive. Some forms of breast cancer are resistant to conventional therapies. It is important that efficacious treatments are selected before the cancer has an opportunity to metastasize. Improved methods for characterizing and treating breast cancer are urgently required.

### SUMMARY OF THE INVENTION

As described below, the present invention features compositions and methods for characterizing the methylation status of the Vitamin D Receptor (VDR) in neoplasia (e.g., breast carcinoma), selecting an appropriate therapy, and treating the neoplasia (e.g., breast carcinoma).

In one aspect, the invention generally provides a method for characterizing a breast carcinoma in a biologic sample, the method comprising quantifying the promoter methylation of the vitamin D receptor in the sample, wherein an increased quantity of promoter methylation relative to a reference indicates that the breast carcinoma is vitamin D-resistant.

In another aspect, the invention provides a method for detecting a breast carcinoma in a biologic sample, the method comprising quantifying the promoter methylation of the vitamin D receptor in the sample, wherein an increased quantity of promoter methylation relative to a reference indicates the presence of a neoplasia in the sample.

In yet another aspect, the invention provides a method of selecting a treatment for a subject diagnosed as having breast carcinoma, the method involving quantifying the level of vitamin D receptor promoter methylation in a biologic sample from the subject relative to a reference, wherein the level of promoter methylation is indicative of a treatment; and selecting a treatment. In one embodiment, an increase in promoter methylation indicates the subject should be treated with Calcitriol and a demethylating agent or HDAC inhibitor.

In yet another aspect, the invention provides a method of monitoring a subject diagnosed as having a breast carcinoma, the method comprising quantifying the level of vitamin D receptor promoter methylation in a sample derived from the subject, wherein an altered level of promoter methylation relative to the level of methylation in a reference indicates an altered severity of carinoma in the subject. In one embodiment, the reference is the level of methylation present in a sample previously obtained from the subject. In another embodiment, the reference is a baseline level of methylation present in a sample from the subject obtained prior to therapy. In another embodiment, the reference is the level of methylation present in a normal patient sample. In another embodiment, a reduced level of promoter methylation indicates a reduced severity of neoplasia. In another embodiment, detection of no alteration in the level of promoter methylation indicates no reduction in the severity of the neoplasia.

In yet another aspect, the invention provides a method of identifying a subject as having a propensity to develop a breast carcinoma, the method comprising obtaining a breast tissue sample from the subject, quantifying the level of vitamin D receptor promoter methylation in the sample, wherein an altered level of promoter methylation relative to the level of methylation in a reference identifies the subject as having a propensity to develop a breast carcinoma. In one embodiment, the tissue sample comprises a precancerous lesion. In another embodiment, the precancerous lesion is selected from the group consisting of simple hyperplasia, atypical hyperplasia, and breast carcinoma in. In another embodiment, the tissue sample is obtained in as a core biopsy or fine needle aspirant.

In another aspect, the invention provides a method of treating or preventing vitamin D-resistant breast carcinoma in a subject, the method comprising administering to the subject an effective amount of Calcitriol and a demethylating agent or an HDAC inhibitor. In one embodiment, the histone deacetylase inhibitor is selected from the group consisting of Scriptaid, varinostat, APHA Compound 8, Apicidin, sodium butyrate, (−)-Depudecin, HMBA, valproic acid, Sirtinol, trichostatin A, and salts or analogs thereof. In another embodiment, the demethylating agent sensitizes cells to Calcitriol.

In another aspect, the invention provides a kit for the analysis of promoter methylation, the kit comprising at least one primer capable of distinguishing between methylated and unmethylated promoter, and directions for using the primer for the analysis of promoter methylation.

In another aspect, the invention provides a kit for the analysis of promoter methylation, the kit comprising primers useful for bisulfite sequencing, and directions for using the primers for the analysis of vitamin D receptor promoter methylation.

In another aspect, the invention provides a kit for the analysis of vitamin D receptor promoter methylation, the kit comprising at least one primer capable of distinguishing between methylated and unmethylated vitamin D receptor promoter, and directions for using the primer for the analysis of promoter methylation. In one embodiment, the aforementioned kits further contain a pair of primers a reference gene.

In yet another aspect, the invention provides a collection of primer sets, each of the primer sets comprising at least two (e.g., 2, 3, 4, 5, 6, 7, or more) primers that bind to vitamin D receptor promoter, wherein at least one of the primers is capable of distinguishing between methylated and unmethylated vitamin D receptor promoter.

In yet another aspect, the invention provides a collection of primer sets, wherein each of the primer sets comprises at least two primers capable of amplifying a sequence comprising a methylated region of the vitamin D receptor promoter. In one embodiment, the aforementioned collections contain a primer having a sequence shown in Table 1.

In another aspect, the invention provides a pharmaceutical composition comprising an effective amount of calcitriol and a HDAC inhibitor or demethylating agent in a pharmaceutically acceptable excipient. In one embodiment, the demethylating agent is 5-azacyitidine and the histone deacetylase inhibitor is any one or more of Scriptaid, varinostat (e.g., SAHA), APHA Compound 8, Apicidin, sodium butyrate, (−)-Depudecin, HMBA, valproic acid, Sirtinol, trichostatin A, and salts or analogs thereof.

In yet another aspect, the invention provides a method for characterizing a breast carcinoma, the method comprising detecting the expression of one or more vitamin D receptor variants in the sample, wherein detection of an increased number of such variants relative to a reference indicates that the breast carcinoma is vitamin D-resistant.

In yet another aspect, the invention provides a method for detecting a breast carcinoma in a biologic sample, the method comprising detecting the expression of a vitamin D receptor or variants thereof in the sample, wherein detection of an increased level of total vitamin D receptor transcripts or variants thereof relative to a reference indicates the presence of a carcinoma in the sample. In one embodiment, the method detects 5′ splice variants.

In another aspect, the invention provides a collection of primers that amplifies a sequence encoding a vitamin D receptor transcript or variant thereof. In one embodiment, the collection comprises a primer having a sequence shown in Table 1.

In various embodiments of any of the above aspects, promoter methylation is quantified using bisulfite sequencing, QMSP, or any other method delineated herein. In certain embodiments of any of the above aspects, sequencing is performed between nucleic acids 790 bp upstream and 380 bp downstream of the VDR transcription start site. In other embodiments of any of the above aspects, the primers interrogate areas of high methylation between about −760 and −450. In other embodiments of any of the above aspects, the method further involves measuring the expression of a VDR downstream gene selected from the group consisting of CYP27B1, CYP24A1 CYP3A4 and p21. In still other embodiments, the method detects an increase in one or more of CYP27B1, CYP24A1 and p21 in cancer tissue relative to normal tissue. In still other embodiments, the method detects a decrease in CYP3A4 in cancer tissue relative to normal tissue. In still other embodiments, the reference is the level of methylation present at the promoter in a control sample. In still other embodiments, the control sample is derived from a healthy subject. In still other embodiments, the promoter methylation is quantified using quantitative methylation-specific PCR (QMSP). In still other embodiments, the biologic sample is a patient sample. In still other embodiments, the method detects a hypermethylated region. In still other embodiments, detection of a hypermethylated region identifies the breast carcinoma as vitamin D-resistant. In other embodiments of any of the above aspects, an increase in promoter methylation indicates the subject should be treated with Calcitriol and a demethylating agent or HDAC inhibitor. In another embodiment, the reference is the level of methylation present in a normal patient sample.

The invention provides features compositions and methods for characterizing the methylation status of the Vitamin D Receptor in neoplastic tissues, including breast carcinomas. Compositions and articles defined by the invention were isolated or otherwise manufactured in connection with the examples provided below. Other features and advantages of the invention will be apparent from the detailed description, and from the claims.

### DEFINITIONS

By “Vitamin D receptor (VDR)” is meant an intracellular hormone receptor polypeptide r fragment thereof that specifically binds the active form of vitamin D (1,25-dihydroxyvitamin D3 or calcitriol) and/or interacts with target-cell nuclei to produce a variety of biologic effects. A nucleic acid sequence encoding a VDR polypeptide is described by Baker et al., Proc Natl Acad Sci USA. 1988 May; 85(10):3294-8. In one embodiment, the VDR is encoded by a nucleic acid sequence defined by GenBank Accession No. NC_{—}000012.11. In another embodiment, the VDR polypeptide is encoded by GenBank Accession No. J03258.

By “Vitamin D receptor promoter” is meant a regulatory sequence that directs expression of the vitamin D receptor. In one embodiment, the vitamin D receptor promoter comprises at least a portion of a sequence shown at FIG. 3. The following nucleic acid sequence includes nucleic acid sequences represented schematically in FIG. 3.

VDR Sequence 1-1171 (−789 to +380 of TS)
TGGTGAGTTTTCCGCAGCCATCCACAATTCCAGGTCTCAGGAGGTAGTC
TTTATCTTTTCCCCTCACGCCGATGCCACGGGGCGGGGGGGGAAGGCGG
AACTCGGGACCAGGGACCAGGGAAGCTGAGACTCAGCTCCCTTGGGTGA
GATTCGCGACAGGCCGGGAACGTGGTCAGCCGCGGTCGCTGCCAAGGTG
ATATCGGGTGGGAGCAATGACGCAACTCCGGTTTCCACTTCGGCCCCCC
GGGATATTTTACCCTAATCTGTGGGATCAGGCTGAGCTTCCTGGCGTTC
TGCAGCAGTAACAGGTTGGCGAGCGGAGCCCGGGATTTCCCATTCGTGC
GGAGCTAGCCGCCGGTGCCAGTCGGCAGGCGCCCCCCAGCGTCCCGCGG
ACGACGAAGTCCTGGCCTGGTCAGCCCAGGTGGGGGTGACGCACCTGGC
TCAGGCGTCCGCAGCAGGCTGGGTAGAACCACGGCAGGAAGGGTGGGGG
GCTGCATCCCCGATTAACACAGGCTGAAGCGGGTATCCGCACCTATAAT
CATCGACAACTCTGTCCCACAGAGGGCAGAAGCGTGCCTTGCCCTATGG
ACGACGGTCGATGAAAATTTCACGAGTTAGAGTATCTAAGGCTACAGCG
TGGCCTATAGGGTGGTTGATTCCAAGTCAAGATGGTTGCAGCGCCAACG
GAGCTCCTGGCAAGAGAGGACTGGACCTGTGGGCGGGGCGGAGGGGCGG
GGCGGGGCCGGGGCGGGGCCTGACCGAGAGGCGGGGCCAGGTGCTGGGC
TGTCTCTGCTTGTCAAAAGGCGGCAGCGGAGCCGTGTGCGCCGGGAGCG
CGGAACAGCTTGTCCACCCGCCGGCCGGACCAGGTGCGAACCCGGGAGC
AGCGGGAAAGGGGGTCTCAGGATAGGGACTCGGGGTCGGGGCGTCTGGG
ATACCGGGGCCTGAGCGCCCGGCTGCGAGCATTAGAGTCTAAGTCTCAG
CGGTAAACTTGGCTACTGAGGTCCGGGCTGTCGTGCCATGAGGCTGGGA
CACTAAGGGGCACTGAGGTTTGAGAAAGCTGAAGTTCGTGCCAGGCTGG
CGAGGGGAGCAGCGACATCCTCGGCGCTTAGGAGAAATGCTCCGCTAAC
ACAGTGCTTAGCACTTGGGCAACAAGAAGTATTTGTTTCCTTCT.

In another embodiment, the promoter sequence comprises at least about nucleic acid positions −789 to +380 relative to the transcription start site of VDR (NT_{—}029419.12: c10442909<-10441739=1->1170).

The term “Vitamin D resistant” refers to a cell that fails to respond or that shows a reduced response to an amount of Vitamin D capable of inducing a response in a reference cell or tissue; or a cell that requires an increased amount of vitamin D to generate a comparable response.

By “demethylating agent” is meant an agent that demethylates DNA. In one embodiment, the demethylating agent inhibits DNA methyl transferase activity.

By “histone deacetylase (HDAC) inhibitor” is meant an agent that increases histone acetylation. In one embodiment, the HDAC inhibitor increases histone acetylation thereby favoring transcription.

By “alteration” is meant an increase or decrease. An alteration may be by as little as 1%, 2%, 3%, 4%, 5%, 10%, 20%, 30%, or by 40%, 50%, 60%, or even by as much as 75%, 80%, 90%, or 100%.

By “biologic sample” is meant any tissue, cell, fluid, or other material derived from an organism. In one embodiment, a biological sample is a breast carcinoma biopsy (e.g., a needle biopsy).

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

By “control” is meant a standard of comparison. For example, the methylation level present at a promoter in a neoplasia may be compared to the level of methylation present at that promoter in a corresponding normal tissue.

By “diagnostic” is meant any method that identifies the presence of a pathologic condition or characterizes the nature of a pathologic condition (e.g., a neoplasia). Diagnostic methods differ in their sensitivity and specificity. While a particular diagnostic method may not provide a definitive diagnosis of a condition, it suffices if the method provides a positive indication that aids in diagnosis.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include breast carcinoma and precancerous lesions of the breast (e.g., simple hyperplasia, atypical hyperplasia, and breast carcinoma in situ), as well as other neoplasias.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “increased methylation” is meant a detectable positive change in the level, frequency, or amount of methylation. Such an increase may be by 5%, 10%, 20%, 30%, or by as much as 40%, 50%, 60%, or even by as much as 75%, 80%, 90%, or 100%.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

By “periodic” is meant at regular intervals. Periodic patient monitoring includes, for example, a schedule of tests that are administered daily, bi-weekly, bi-monthly, monthly, bi-annually, or annually.

By “promoter” is meant a nucleic acid sequence sufficient to direct transcription. In general, a promoter includes, at least, 50, 75, 100, 125, 150, 175, 200, 250, 300, 400, 500, 750, 1000, 1500, or 2000 nucleotides upstream of a given coding sequence.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “severity of neoplasia” is meant the degree of pathology. The severity of a neoplasia increases, for example, as the stage or grade of the neoplasia increases.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100.mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 is a schematic diagram showing splice variant-selective primer design and detailed structure of the 5′VDR gene locus exons. The V1 primers detect three VDR variant products (V1, V2, V3) spanning the 5′ flanking region of exon 1a to the end of exon 2. Variant V1 (167 bp product) lacks exons 1d and 1b. Variant V2 (290 bp product) is devoid of exon 1d and Variant V3 (85 bp) is devoid of exons 1d, 1b and 1c. V1 and V2 both encode the same active 427 amino acid VDRA protein. The V1d primers detect three VDR variant products (V1d, V1d′ and V1d″) extending from the 5′ flanking region of exon1d to exon2. V1d′ (279 bp product) contains both exon1b and exon1c, V1d (156 bp product) is lacking exon1b, and V1d″ (87 bp product) contains neither exon 1b nor exon1c. Variants V1d and V1d″ encode active VDR proteins of 477 amino acids (VDRB1) and 450 amino acids (VDRB2), respectively. The V primers detect all of the above VDR variants (VT), and span exons 3 and 4, producing a 223 bp product. The second column depicts the variants that were identified in breast cancer tissue by cloning and sequencing the gel purified RT-PCR products; the third column lists the predicted VDR peptides. Alternative translational start sites are indicated with a * at the top of exon 1d (coding for VDRB proteins) and exon 2 (coding for VDRA proteins).

FIGS. 2A and 2B are graphs showing that demethylation potentiates Calcitriol-induced growth inhibition and VDR Expression. The immortalized normal breast cell line HBL100 and the breast cancer cell lines HS578T, 21PT, MCF-7, and T47D were treated with AZA (5-azacyitidine; 5 μM), Calcitriol (D; 1.5 μM), alone or in combination and compared to vehicle control (CON). After 96 hours, growth inhibition (A) and VDR expression (B) was assessed. The effects observed were highly significant (* p<0.05; ** p<0.01). FIG. 2A shows the results of an MMT assay. Within each cell line, the percentage of viable cells recovered after each drug treatment is indicated. The mean and standard deviation of triplicate experiments are shown. FIG. 2B shows the results of a Quantitative RT-PCR assay of total VDR expression. Expression levels of VDR are expressed as ratio of inverse Ct values of VDR to b-actin transcripts. The V and BA primer sets were used to assess total VDR and beta actin, respectively.

FIG. 3 shows the results of bisulfite sequencing of the VDR promoter in breast cell lines and tissues. Nucleotide positions of the VDR promoter region are indicated relative to the transcriptional start site of exon 1a (TS). Grey, black, striped and white vertical bars at the top represent DNA binding sites of Spl, NF-kB, AP-1 and AP-2 transcription factors, respectively. Individual CpG dinucleotide methylation levels are shown as average of three independent reads, where white circles indicate 0-25% methylation, white-black circles indicate 25-50% methylation, and black circles indicate 50-100% methylation. For cell lines, 1, 2 and 3 represent untreated control cells, Calcitriol-treated and AZA-treated cells, respectively. The sequences in the lower half were derived from 8 breast cancers, 7 adjacent normal breast tissue samples, and 3 organoid preparations from normal breast tissue. The arrowheads indicate the locations of the primers used for the MSP assays.

FIG. 4 is a quantification of VDR methylation. A nested PCR approach was used to assess VDR methylation in tissue samples. Bisulfite-treated DNA was pre-amplified with the P1 sequencing primers, followed by a qMSP reaction using the M and UN primers. The fraction of methylated VDR promoter is expressed as percent methylation level in 15 breast cancers and 7 normal breast tissues. Round symbols indicate DNA obtained from fresh frozen tissue, squares indicate FFPEderived DNA. The box plots show significantly higher VDR methylation in breast cancers than normal tissue controls (p<0.0002 by Wilcoxon rank sum test).

FIGS. 5A-5D show that different VDR variants are present in breast cancer tissue and normal breast tissue organoids. FIG. 5A shows the results of an agarose gel electrophoresis. Shown are the products of reverse-transcriptase PCR amplifications using V1 (top gel) and V1d primers (bottom gel) in breast cancer tissues (Cancer) and in normal breast organoids (Normal). Standard qRT-PCR conditions (40 cycles) were used. See FIG. 1 for details of the variants detected. β-actin transcript levels are shown below. FIGS. 5B and 5C show quantitation of VDR transcripts at limiting PCR cycle number. FIG. 5B is an agarose gel electrophoresis showing the products of reverse transcriptase-PCR amplifications using V1 and V1d primers is shown on the left. Semi-quantitative PCR reaction conditions (28 cycles) were used in order to remain in the linear product range of the amplifications. The bars in the matching graph on the right represent ratios of VDR variants to beta actin, in cancer tissue samples (grey) and in normal breast organoids (white). The right-most bars (VT) reflect the results obtained using the V primers that detect exon 3 & 4 transcripts. Means and standard deviations of triplicate experiments are shown. Significant differences between normal and cancer are indicated (* p<0.05; **p<0.01). FIG. 5D is a graph showing a Quantitation of VDR variant expression after Calcitriol and AZA treatment. Breast cell lines were treated as in FIG. 2A. Reverse transcriptase-PCR reactions were performed with the V1 and V1d primer sets to identify variant expression levels. GAPDH was used to normalize VDR expression levels. The bar graphs summarize our results, with means and standard deviations of the five cell lines shown.

Significant differences compared to vehicle-only controls (fold induction=1) are indicated (* p<0.05; ** p<0.01).

FIGS. 6A-6C show expression levels of VDR Response Element (VDRE)-containing genes in primary breast cancer and normal breast tissue. FIG. 6A shows the results of an assessment of VDRE-containing gene expression in vivo using agarose gel electrophoresis of RT-PCR of CYP3A4 (24/25-OH), CYP27B1 (1-OH), CYP24A1 (24-OH) and p21. Assays were performed in triplicate on a total of 8 breast cancers and 3 normal organoid preparations. FIG. 6B shows the quantitation of VDRE-containing gene expression in vivo. The matching bar graph shows ratios of individual gene transcripts to beta actin, in cancer tissue (grey) and in normal breast organoids (white). Means and standard deviations are shown. Significant differences between normal and cancer are indicated (* p<0.05; ** p<0.01). FIG. 6C shows the induction of VDRE-containing gene expression after Calcitriol and AZA treatment in vitro. The bars represent the fold induction of VDR downstream gene targets (CYP3A4[24/25OH]-white, CYP27B1[1OH]-grey, and C/EBP-hatched) over untreated control (horizontal line=1.0). Means and standard deviations of triplicate experiments are shown (* p<0.05; ** p<0.01).

### DETAILED DESCRIPTION OF THE INVENTION

The invention features compositions and methods that are useful for characterizing a neoplasia, selecting a treatment, and treating the neoplasia.

The invention is based, at least in part, on the discovery that the Vitamin D receptor promoter is aberrantly methylated in breast cancer and that methylation mediated silencing of expression of the functional variants of the VDR may contribute to reduced expression of downstream effectors of the VDR pathway and subsequent Calcitriol insensitivity in breast cancer. These data suggest that pharmacological reversal of VDR methylation will likely re-establish breast cancer cell susceptibility to differentiation therapy using Calcitriol analogs.

As reported in more detail below, the resistance mechanism and the role of epigenetic silencing of VDR by promoter hypermethylation were investigated. Bisulfite sequencing of the VDR promoter region revealed methylated CpG islands at −700 base pairs (bp) upstream and near the transcription start site. VDR CpG islands were demethylated by 5′ deoxy-azacytidine (5′ dAZA) treatment, and this was accompanied by a parallel increase in VDR mRNA levels in breast cancer cell lines. Quantitative methylationspecific PCR analyses confirmed the absence of VDR methylation in normal breast tissue (0/5), and presence of methylation in primary tumors (15/15) in these islands. Truncated, potentially inactive, VDR transcripts were detected in breast cancers, but not in normal breast tissue. Consistent with this observation, VDR-responsive genes, such as cytochrome p450 hydroxylases and p21, were underexpressed in breast cancers compared to normal breast samples. Expression of the active longer transcripts of VDR was restored with 5′ dAZA treatment, with a concurrent increase in expression of VDRE containing genes.

### Calcitriol

Certain biological and epidemiological data suggest that Calcitriol, the active form of Vitamin D (1α, 25(OH)₂-Vitamin D3, VTD), may play an important role in cancer prevention

(Deeb et al., Nat Rev Cancer 2007; 7:684-700; Thorne et al., The Proceedings of the Nutrition Society 2008; 67:115-27; Raimondi et al., Carcinogenesis 2009). Calcitriol regulates proliferation and induces differentiation of a wide variety of cells, including the normal mammary gland (Welsh et al., J Steroid Biochem Mol Biol 2002; 83:85-92; Colston et al., Endocr Relat Cancer 2002; 9:45-59). The biological effects of Calcitriol are mediated through the vitamin D receptor (VDR), a nuclear transcription factor that binds to the Vitamin D responsive element (VDRE) present in the promoters of genes responsive to Calcitriol. Immunohistochemical data show that VDR expression is higher in differentiated cells than in proliferating cells, and that the Calcitriol signaling pathway participates in negative growth regulation of the mammary gland (Zinser et al., Development 2002; 129:3067-76). Furthermore, VDR knockout mice show impaired ductal differentiation and branching in the mammary gland compared to wild type littermates (Zinser et al., Development 2002; 129:3067-76).

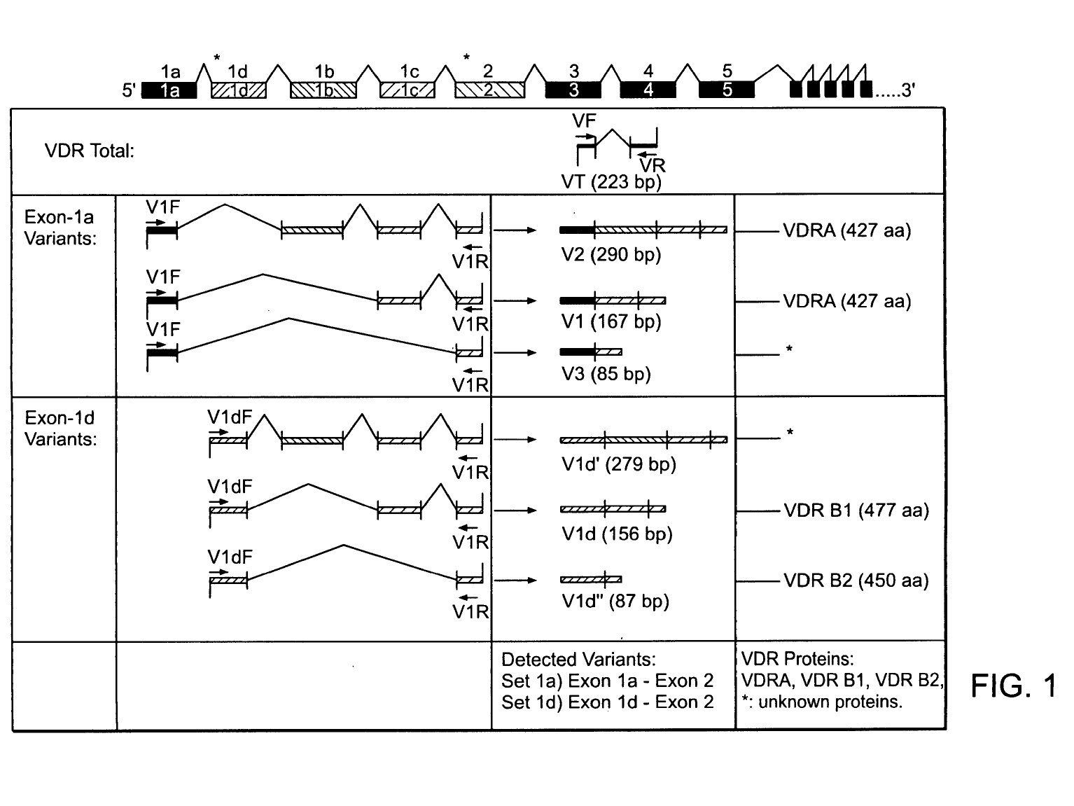

Attempts to use Calcitriol therapeutically have been uniformly disappointing because many cancers display vitamin D insensitivity. VDR is not commonly mutated in cancer, and the reasons for this insensitivity remain largely unexplored. Most reports have focused on altered patterns of histone acetylation in an attempt to explain this insensitivity (Banwell et al., Recent Results Cancer Res 2003; 164:83-98). However, a few studies have documented aberrant DNA methylation patterns in the VDR promoter in colon cancer and endometrial cancer (Smirnoff et al., Oncol Res 1999; 11:255-64; Whitcomb et al., Clin Cancer Res 2003; 9:2277-87). An in-silico analysis of the VDR gene, focusing on the evolutionarily well-conserved exons 1a and 1d (see FIG. 1) identified three CpG islands in an area spanning from −790 bp upstream to +380 bp downstream relative to the primary VDR transcription start site in exon 1a. This region contains regulatory elements, including several SP1 and AP-2 sites, where methylation may affect the binding of transcription factors. As reported in more detail below, the results reported herein indicate that methylation-induced silencing of VDR transciption in breast cancer accounts for Calcitriol insensitivity, and that this insensitivity could be reversed using demethylating agents, thus mitigating Calcitriol resistance in breast cancer cells.

### Diagnostic Assays

The present invention provides a number of diagnostic assays that are useful for the identification or characterization of a carcinoma (e.g., breast cancer). In one embodiment, a neoplasia is characterized by quantifying or determining the methylation level of Vitamin D receptor promoter in the carcinoma. In one embodiment, methylation levels are determined using bisulfite sequencing or quantitative methylation specific PCR (QMSP) to detect CpG methylation in genomic DNA. QMSP uses sodium bisulfate to convert unmethylated cytosine to uracil. A comparison of sodium bisulfate treated and untreated DNA provides for the detection of methylated cytosines.

While the examples provided below describe methods of detecting methylation levels using bisulfite sequencing, the skilled artisan appreciates that the invention is not limited to such methods. Methylation levels are quantifiable by any standard method, such methods include, but are not limited to QMSP, real-time PCR, bisulfite genomic DNA sequencing, restriction enzyme-PCR, MSP (methylation-specific PCR), methylation-sensitive single nucleotide primer extension (MS-SNuPE) (see, for example, Kuppuswamy et al., Proc. Natl. Acad. Sci. USA, 88, 1143-1147, 1991), DNA microarray based on fluorescence or isotope labeling (see, for example, Adorján Nucleic Acids Res., 30: e21 and Hou Clin. Biochem., 36:197-202, 2003), the Infinium Methylation Assay™, which provides a commercially available array-based methylation assay (Illumina Inc., San Diego, Calif.), mass spectroscopy, methyl accepting capacity assays, and methylation specific antibody binding. See also U.S. Pat. Nos. 5,786,146, 6,017,704, 6,300,756, and 6,265,171.

The primers used in the invention for amplification of the methylated promoter in the sample specifically distinguish between methylated, and non-methylated DNA. Methylation specific primers for the non-methylated DNA preferably have a T in the 3′ CG pair to distinguish it from the C retained in methylated DNA, and the complement is designed for the antisense primer.***] The primers of the invention embrace oligonucleotides of sufficient length and appropriate sequence so as to provide specific initiation of polymerization on a significant number of nucleic acids. Specifically, the term “primer” as used herein refers to a sequence comprising two or more deoxyribonucleotides or ribonucleotides, preferably more than three, and most preferably more than 8, which sequence is capable of initiating synthesis of a primer extension product, which is substantially complementary to a polymorphic locus strand. The primer must be sufficiently long to prime the synthesis of extension products in the presence of the inducing agent for polymerization. The exact length of primer will depend on many factors, including temperature, buffer, and nucleotide composition. The oligonucleotide primer typically contains between 12 and 27 (e.g., 12, 13, 14, 15, 16, 17, 18, 19, 20, 21, 22, 23, 24, 25, 26, 27) nucleotides or more nucleotides, although it may contain fewer nucleotides. Preferably, an oligonucleotide primer contains 15, 16, 17, 18, 19, or 20 nucleotides.

Primers of the invention are designed to be “substantially” complementary to each strand of the genomic locus to be amplified. This means that the primers must be sufficiently complementary to hybridize with their respective strands under conditions that allow the agent for polymerization to perform. In other words, the primers should have sufficient complementarity with the 5′ and 3′ flanking sequences to hybridize therewith and permit amplification of the genomic locus. While exemplary primers are provided herein, it is understood that any primer that hybridizes with the target sequences of the invention are useful in the method of the invention for detecting methylated nucleic acid.

In one embodiment, methylation specific primers amplify a desired genomic target using the polymerase chain reaction (PCR). The amplified product is then detected using standard methods known in the art. In one embodiment, a PCR product (i.e., amplicon) or real-time PCR product is detected by probe binding. In one embodiment, probe binding generates a fluorescent signal, for example, by coupling a fluorogenic dye molecule and a quencher moiety to the same or different oligonucleotide substrates (e.g., TaqMan® (Applied Biosystems, Foster City, Calif., USA), Molecular Beacons (see, for example, Tyagi et al., Nature Biotechnology 14(3):303-8, 1996), Scorpions® (Molecular Probes Inc., Eugene, Oreg., USA)). In another example, a PCR product is detected by the binding of a fluorogenic dye that emits a fluorescent signal upon binding (e.g., SYBR® Green (Molecular Probes)). Such detection methods are useful for the detection of a methylation specific PCR product.

The methylation level of any two or more of the promoters described herein defines the methylation profile of a carcinoma. The level of methylation present at the Vitamin D receptor promoter is compared to a reference. In one embodiment, the reference is the level of methylation present in a control sample obtained from a patient that does not have a carcinoma. In another embodiment, the reference is a baseline level of methylation present in a biologic sample derived from a patient prior to, during, or after treatment for a carcinoma. In yet another embodiment, the reference is a standardized curve.

The methylation level of the Vitamin D receptor promoter described herein is used, alone or in combination with other methods, to characterize the breast carcinoma. In one embodiment the carcinoma is characterized to determine its stage or grade. Grading is used to describe how abnormal or aggressive the neoplastic cells appear, while staging is used to describe the extent of the neoplasia.

### Selection of a Treatment Method

After a subject is diagnosed as having a carcinoma (e.g., breast cancer) or the carcinoma is characterized, a method of treatment is selected. In vitamin-D resistant breast cancer, for example, treatment is commenced with calcitriol and a demethylating agent (e.g., 5′ deoxy-azacytidine or an HDAC inhibitor). Such treatment may be combined with any one or a number of standard treatment regimens. The methylation profile of the neoplasia, or the level of methylation at the Vitamin D receptor promoter, is used in selecting a treatment method.

### Histone Deacetylase Inhibitors

Regulation of gene expression is mediated by several mechanisms, including the post-translational modifications of histones by dynamic acetylation and deacetylation. The enzymes responsible for reversible acetylation/-deacetylation processes are histone acetyltransferases (HATs) and histone deacetylases (HDACs), respectively. Histone deacetylase inhibitors include Scriptaid, varinostat (e.g., SAHA), APHA Compound 8, Apicidin, sodium butyrate, (−)-Depudecin, HMBA, valproic acid, Sirtinol, trichostatin A, and salts or analogs thereof.

### Pharmaceutical Compositions

The present invention features pharmaceutical preparations comprising compounds together with pharmaceutically acceptable carriers, where the compounds provide for the demethylation of a hypermethylated vitamin D promoter that increases sensitivity to calcitriol. Such preparations have both therapeutic and prophylactic applications. In one embodiment, a pharmaceutical composition includes a demethylating agent (e.g., HDAC inhibitor) in combination with calcitriol. The demethylating agent (e.g., HDAC inhibitor) and calcitriol are formulated together or separately. In another embodiment, a pharmaceutical composition further includes an agent that is used conventionally for the treatment of cancer (e.g., breast cancer). Compounds of the invention may be administered as part of a pharmaceutical composition. The compositions should be sterile and contain a therapeutically effective amount of the polypeptides in a unit of weight or volume suitable for administration to a subject. The compositions and combinations of the invention can be part of a pharmaceutical pack, where each of the compounds is present in individual dosage amounts.

Pharmaceutical compositions of the invention to be used for prophylactic or therapeutic administration should be sterile. Sterility is readily accomplished by filtration through sterile filtration membranes (e.g., 0.2 μm membranes), by gamma irradiation, or any other suitable means known to those skilled in the art. Therapeutic compositions generally are placed into a container having a sterile access port, for example, an intravenous solution bag or vial having a stopper pierceable by a hypodermic injection needle. These compositions ordinarily will be stored in unit or multi-dose containers, for example, sealed ampoules or vials, as an aqueous solution or as a lyophilized formulation for reconstitution.

The compounds may be combined, optionally, with a pharmaceutically acceptable excipient. The term “pharmaceutically-acceptable excipient” as used herein means one or more compatible solid or liquid filler, diluents or encapsulating substances that are suitable for administration into a human. The term “carrier” denotes an organic or inorganic ingredient, natural or synthetic, with which the active ingredient is combined to facilitate administration. The components of the pharmaceutical compositions also are capable of being co-mingled with the molecules of the present invention, and with each other, in a manner such that there is no interaction that would substantially impair the desired pharmaceutical efficacy.

Compounds of the present invention can be contained in a pharmaceutically acceptable excipient. The excipient preferably contains minor amounts of additives such as substances that enhance isotonicity and chemical stability. Such materials are non-toxic to recipients at the dosages and concentrations employed, and include buffers such as phosphate, citrate, succinate, acetate, lactate, tartrate, and other organic acids or their salts; tris-hydroxymethylaminomethane (TRIS), bicarbonate, carbonate, and other organic bases and their salts; antioxidants, such as ascorbic acid; low molecular weight (for example, less than about ten residues) polypeptides, e.g., polyarginine, polylysine, polyglutamate and polyaspartate; proteins, such as serum albumin, gelatin, or immunoglobulins; hydrophilic polymers, such as polyvinylpyrrolidone (PVP), polypropylene glycols (PPGs), and polyethylene glycols (PEGs); amino acids, such as glycine, glutamic acid, aspartic acid, histidine, lysine, or arginine; monosaccharides, disaccharides, and other carbohydrates including cellulose or its derivatives, glucose, mannose, sucrose, dextrins or sulfated carbohydrate derivatives, such as heparin, chondroitin sulfate or dextran sulfate; polyvalent metal ions, such as divalent metal ions including calcium ions, magnesium ions and manganese ions; chelating agents, such as ethylenediamine tetraacetic acid (EDTA); sugar alcohols, such as mannitol or sorbitol; counterions, such as sodium or ammonium; and/or nonionic surfactants, such as polysorbates or poloxamers. Other additives may be included, such as stabilizers, anti-microbials, inert gases, fluid and nutrient replenishers (i.e., Ringer's dextrose), electrolyte replenishers, and the like, which can be present in conventional amounts.

The compositions, as described above, can be administered in effective amounts. The effective amount will depend upon the mode of administration, the particular condition being treated and the desired outcome. It may also depend upon the stage of the condition, the age and physical condition of the subject, the nature of concurrent therapy, if any, and like factors well known to the medical practitioner. For therapeutic applications, it is that amount sufficient to achieve a medically desirable result.

With respect to a subject having a neoplasia, such as breast carcinoma, an effective amount is sufficient to reduce the degree of vitamin D receptor promoter methylation or to increase calcitriol sensitivity. With respect to a subject having a disease or disorder related to promoter hypermethylation, an effective amount is an amount sufficient to stabilize, slow, or reduce a symptom associated with a pathology. Generally, doses of the compounds of the present invention would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of a composition of the present invention.

A variety of administration routes are available. The methods of the invention, generally speaking, may be practiced using any mode of administration that is medically acceptable, meaning any mode that produces effective levels of the active compounds without causing clinically unacceptable adverse effects. In preferred embodiments, a composition of the invention is administered locally or systemically. Other modes of administration include oral, rectal, topical, intraocular, buccal, intravaginal, intracisternal, intracerebroventricular, intratracheal, nasal, transdermal, within/on implants, or parenteral routes. The term “parenteral” includes subcutaneous, intrathecal, intravenous, intramuscular, intraperitoneal, or infusion. Intravenous or intramuscular routes are not particularly suitable for long-term therapy and prophylaxis. They could, however, be preferred in emergency situations. Oral administration can be preferred for prophylactic treatment because of the convenience to the patient as well as the dosing schedule.

Pharmaceutical compositions of the invention can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions of the invention can also contain one or more osmotic modulating agents, i.e., a compound that modulates the osmotic properties (e.g, tonicity, osmolality and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

Compositions comprising a compound of the present invention can contain multivalent metal ions, such as calcium ions, magnesium ions and/or manganese ions. Any multivalent metal ion that helps stabilizes the composition and that will not adversely affect recipient individuals may be used. The skilled artisan, based on these two criteria, can determine suitable metal ions empirically and suitable sources of such metal ions are known, and include inorganic and organic salts.

Pharmaceutical compositions of the invention can also be a non-aqueous liquid formulation. Any suitable non-aqueous liquid may be employed, provided that it provides stability to the active agents (s) contained therein. Preferably, the non-aqueous liquid is a hydrophilic liquid. Illustrative examples of suitable non-aqueous liquids include: glycerol; dimethyl sulfoxide (DMSO); polydimethylsiloxane (PMS); ethylene glycols, such as ethylene glycol, diethylene glycol, triethylene glycol, polyethylene glycol (“PEG”) 200, PEG 300, and PEG 400; and propylene glycols, such as dipropylene glycol, tripropylene glycol, polypropylene glycol (“PPG”) 425, PPG 725, PPG 1000, PPG 2000, PPG 3000 and PPG 4000.

Pharmaceutical compositions of the invention can also be a mixed aqueous/non-aqueous liquid formulation. Any suitable non-aqueous liquid formulation, such as those described above, can be employed along with any aqueous liquid formulation, such as those described above, provided that the mixed aqueous/non-aqueous liquid formulation provides stability to the compound contained therein. Preferably, the non-aqueous liquid in such a formulation is a hydrophilic liquid. Illustrative examples of suitable non-aqueous liquids include: glycerol; DMSO; PMS; ethylene glycols, such as PEG 200, PEG 300, and PEG 400; and propylene glycols, such as PPG 425, PPG 725, PPG 1000, PPG 2000, PPG 3000 and PPG 4000.

Suitable stable formulations can permit storage of the active agents in a frozen or an unfrozen liquid state. Stable liquid formulations can be stored at a temperature of at least −70° C., but can also be stored at higher temperatures of at least 0° C., or between about 0.1° C. and about 42° C., depending on the properties of the composition. It is generally known to the skilled artisan that proteins and polypeptides are sensitive to changes in pH, temperature, and a multiplicity of other factors that may affect therapeutic efficacy.

In certain embodiments a desirable route of administration can be by pulmonary aerosol. Techniques for preparing aerosol delivery systems containing polypeptides are well known to those of skill in the art. Generally, such systems should utilize components that will not significantly impair the biological properties of the antibodies, such as the paratope binding capacity (see, for example, Sciarra and Cutie, “Aerosols,” in Remington's Pharmaceutical Sciences, 18th edition, 1990, pp 1694-1712; incorporated by reference). Those of skill in the art can readily modify the various parameters and conditions for producing polypeptide aerosols without resorting to undue experimentation.

Other delivery systems can include time-release, delayed release or sustained release delivery systems. Such systems can avoid repeated administrations of compositions of the invention, increasing convenience to the subject and the physician. Many types of release delivery systems are available and known to those of ordinary skill in the art. They include polymer base systems such as polylactides (U.S. Pat. No. 3,773,919; European Patent No. 58,481), poly(lactide-glycolide), copolyoxalates, polycaprolactones, polyesteramides, polyorthoesters, polyhydroxybutyric acids, such as poly-D-(−)-3-hydroxybutyric acid (European Patent No. 133, 988), copolymers of L-glutamic acid and gamma-ethyl-L-glutamate (Sidman, K. R. et al., Biopolymers 22: 547-556), poly(2-hydroxyethyl methacrylate) or ethylene vinyl acetate (Langer, R. et al., J. Biomed. Mater. Res. 15:267-277; Langer, R. Chem. Tech. 12:98-105), and polyanhydrides.

Other examples of sustained-release compositions include semi-permeable polymer matrices in the form of shaped articles, e.g., films, or microcapsules. Delivery systems also include non-polymer systems that are: lipids including sterols such as cholesterol, cholesterol esters and fatty acids or neutral fats such as mono- di- and tri-glycerides; hydrogel release systems such as biologically-derived bioresorbable hydrogel (i.e., chitin hydrogels or chitosan hydrogels); sylastic systems; peptide based systems; wax coatings; compressed tablets using conventional binders and excipients; partially fused implants; and the like. Specific examples include, but are not limited to: (a) erosional systems in which the agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480.

Another type of delivery system that can be used with the methods and compositions of the invention is a colloidal dispersion system. Colloidal dispersion systems include lipid-based systems including oil-in-water emulsions, micelles, mixed micelles, and liposomes. Liposomes are artificial membrane vessels, which are useful as a delivery vector in vivo or in vitro. Large unilamellar vessels (LUV), which range in size from 0.2-4.0 μm, can encapsulate large macromolecules within the aqueous interior and be delivered to cells in a biologically active form (Fraley, R., and Papahadjopoulos, D., Trends Biochem. Sci. 6: 77-80).

Liposomes can be targeted to a particular tissue by coupling the liposome to a specific ligand such as a monoclonal antibody, sugar, glycolipid, or protein. Liposomes are commercially available from Gibco BRL, for example, as LIPOFECTIN™ and LIPOFECTACE™, which are formed of cationic lipids such as N-[1-(2,3 dioleyloxy)-propyl]-N,N,N-trimethylammonium chloride (DOTMA) and dimethyl dioctadecylammonium bromide (DDAB). Methods for making liposomes are well known in the art and have been described in many publications, for example, in DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88, 046; EP 143,949; EP 142,641; Japanese Pat. Appl. 83-118008; U.S. Pat. Nos. 4,485,045 and 4,544,545; and EP 102,324. Liposomes also have been reviewed by Gregoriadis, G., Trends Biotechnol., 3: 235-241).

Another type of vehicle is a biocompatible microparticle or implant that is suitable for implantation into the mammalian recipient. Exemplary bioerodible implants that are useful in accordance with this method are described in PCT International application no. PCT/US/03307 (Publication No. WO 95/24929, entitled “Polymeric Gene Delivery System”). PCT/US/0307 describes biocompatible, preferably biodegradable polymeric matrices for containing an exogenous gene under the control of an appropriate promoter. The polymeric matrices can be used to achieve sustained release of the exogenous gene or gene product in the subject.

The polymeric matrix preferably is in the form of a microparticle such as a microsphere (wherein an agent is dispersed throughout a solid polymeric matrix) or a microcapsule (wherein an agent is stored in the core of a polymeric shell). Microcapsules of the foregoing polymers containing drugs are described in, for example, U.S. Pat. No. 5,075,109. Other forms of the polymeric matrix for containing an agent include films, coatings, gels, implants, and stents. The size and composition of the polymeric matrix device is selected to result in favorable release kinetics in the tissue into which the matrix is introduced. The size of the polymeric matrix further is selected according to the method of delivery that is to be used. Preferably, when an aerosol route is used the polymeric matrix and composition are encompassed in a surfactant vehicle. The polymeric matrix composition can be selected to have both favorable degradation rates and also to be formed of a material, which is a bioadhesive, to further increase the effectiveness of transfer. The matrix composition also can be selected not to degrade, but rather to release by diffusion over an extended period of time. The delivery system can also be a biocompatible microsphere that is suitable for local, site-specific delivery. Such microspheres are disclosed in Chickering, D. E., et al., Biotechnol. Bioeng., 52: 96-101; Mathiowitz, E., et al., Nature 386: 410-414.

Both non-biodegradable and biodegradable polymeric matrices can be used to deliver the compositions of the invention to the subject. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene, polyvinylpyrrolidone, and polymers of lactic acid and glycolic acid, polyanhydrides, poly(ortho)esters, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

### Patient Monitoring

The diagnostic methods of the invention are also useful for monitoring the course of a breast carcinoma in a patient or for assessing the efficacy of a therapeutic regimen. In one embodiment, the diagnostic methods of the invention are used periodically to monitor the methylation level of the Vitamin D receptor promoter. In one example, the breast carcinoma is characterized using a diagnostic assay of the invention prior to administering therapy. This assay provides a baseline that describes the methylation level of the Vitamin D receptor promoter prior to treatment. Additional diagnostic assays are administered during the course of therapy to monitor the efficacy of a selected therapeutic regimen. A therapy is identified as efficacious when a diagnostic assay of the invention detects a decrease in methylation levels at the Vitamin D receptor promoter relative to the baseline level of methylation.

### Kits

The invention also provides kits for the diagnosis or monitoring of a breast carcinoma in a biological sample obtained from a subject. In various embodiments, the kit includes at least one primer or probe whose binding distinguishes between a methylated and an unmethylated sequence, together with instructions for using the primer or probe to identify or characterize a breast carcinoma or other neoplasia. In another embodiment, the kit further comprises a pair of primers suitable for use in a polymerase chain reaction (PCR). In yet another embodiment, the kit further comprises a detectable probe. In yet another embodiment, the kit further comprises a pair of primers capable of binding to and amplifying a reference sequence. In yet other embodiments, the kit comprises a sterile container which contains the primer or probe; such containers can be boxes, ampules, bottles, vials, tubes, bags, pouches, blister-packs, or other suitable container form known in the art. Such containers can be made of plastic, glass, laminated paper, metal foil, or other materials suitable for holding nucleic acids. The instructions will generally include information about the use of the primers or probes described herein and their use in diagnosing a neoplasia. Preferably, the kit further comprises any one or more of the reagents described in the diagnostic assays described herein. In other embodiments, the instructions include at least one of the following: description of the primer or probe; methods for using the enclosed materials for the diagnosis of a neoplasia; precautions; warnings; indications; clinical or research studies; and/or references. The instructions may be printed directly on the container (when present), or as a label applied to the container, or as a separate sheet, pamphlet, card, or folder supplied in or with the container.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

It should be appreciated that the invention should not be construed to be limited to the examples that are now described; rather, the invention should be construed to include any and all applications provided herein and all equivalent variations within the skill of the ordinary artisan.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Demethylation Restores the Effects of Calcitriol in Breast Cancer

Calcitriol is a differentiating agent and drugs of this class are known to cause growth arrest and induction of differentiation-associated genes. To test the effect of Calcitriol on breast cancer cells, four breast cancer cell lines (HS578T, 21PT, MCF7, and T47D) and the immortalized normal breast epithelial cell line HBL100 were treated for ninety-six hours with the demethylating agent AZA (5′ deoxy-azacytidine, 10 μM), with Calcitriol (1.5 μM), with Calcitriol+AZA, or with drug vehicle alone. Cell viability was measured by MTT assay.

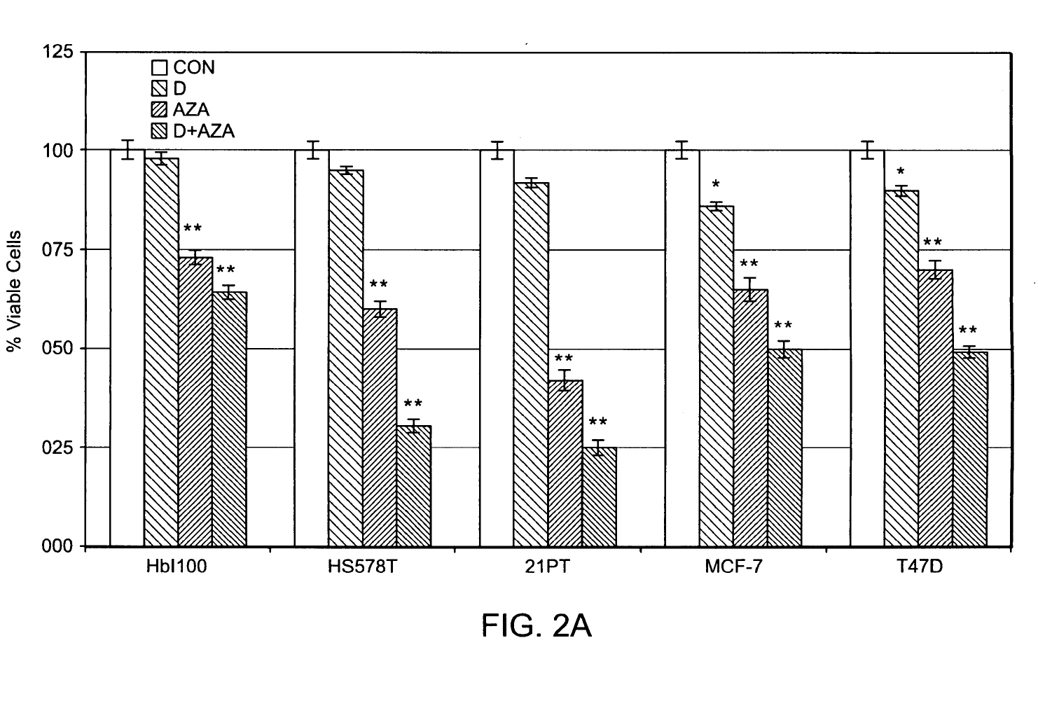

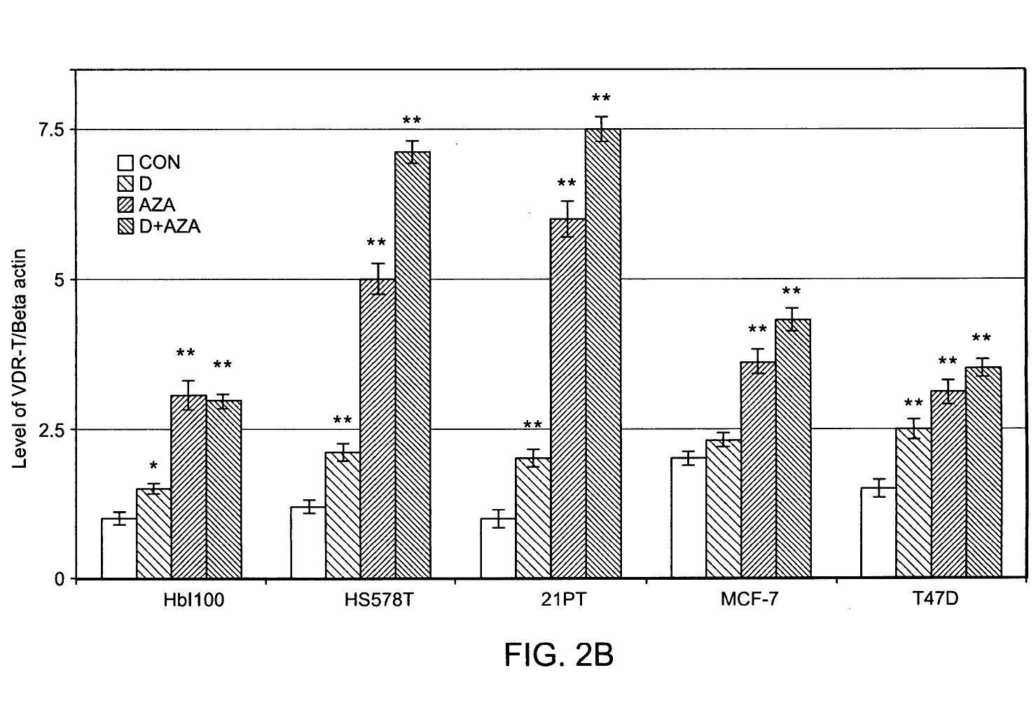

As shown in FIG. 2A, Calcitriol alone had minimal effects on the viability of the five cell lines, compared to vehicle control. The effects of Calcitriol, however, were clearly amplified by AZA in all five cell lines, with stronger antiproliferative effects than with either agent alone. To determine if this correlated with the induction of VDR expression, Quantitative Real-Time PCR (QRT-PCR) analyses were performed using the V primer set designed to detect all VDR transcripts (amplifying regions between VDR exons 2 and 3, see FIG. 1). As shown in FIG. 2B, the overall expression level of VDR increased, with the strongest increase seen when both Calcitriol and AZA were used in combination.

Together these results strongly suggest that sensitivity to Calcitriol is controlled by the level of expression of VDR, which in turn may be regulated by, the status of DNA methylation of the VDR promoter.

### Example 2

### Bisulfite Sequencing of the VDR Promoter Region Reveals Two CpG Hypermethylated Regions in Breast Cancer Cell Lines as Well as Primary Breast Cancer Tissue

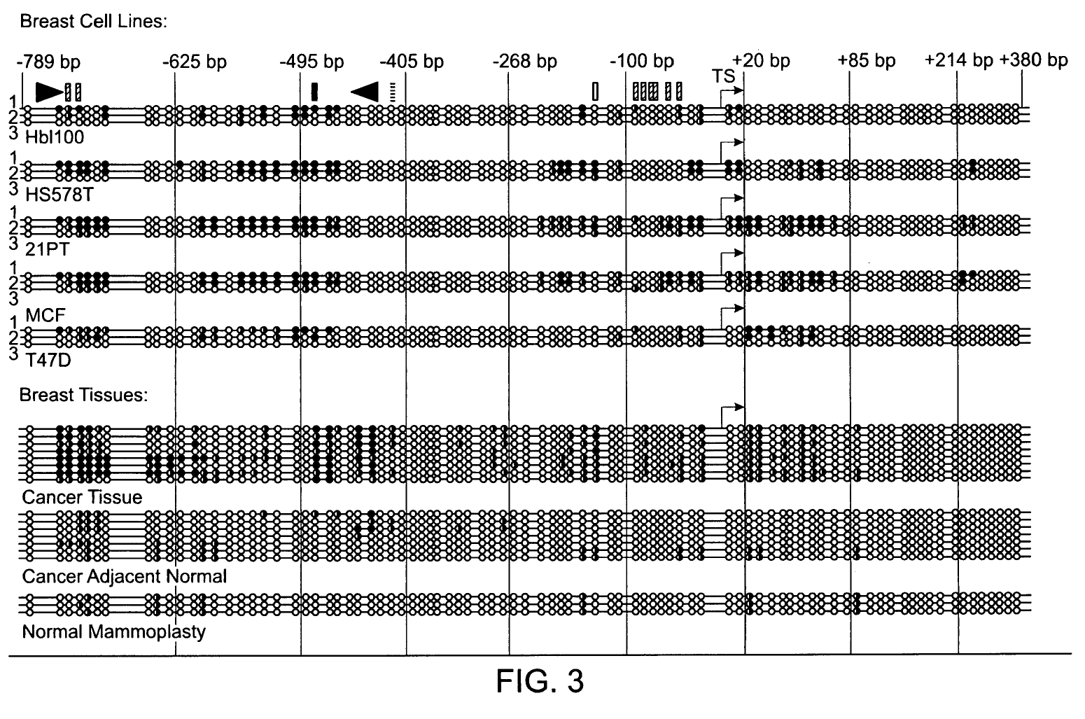

Gene promoter hypermethylation is known to silence gene expression in many cancers. To determine whether the VDR gene promoter was hypermethylated in breast cancer, bisulfite sequencing was performed in the region 790 bp upstream to 380 bp downstream of the VDR transcription start site after treatment of cells in the presence or absence of AZA for ninety six hours (see FIG. 3). Two main regions of AZA-responsive hypermethylation were observed. The first hypermethylated region flanked the upstream pair of SP1-binding sites around −760 bp as well as the NF-κB binding site, and the second flanked the transcriptional start site. Treatment with AZA for ninety six hours induced demethylation of the CpG islands in the VDR promoter in all breast cell lines, whereas Calcitriol alone showed no effect. The extent of hypermethylation was less in HBL100 cells, consistent with their derivation from normal breast tissue. These results were confirmed by methylation specific PCR (MSP).

To validate these in vitro data, DNA from eight freshly frozen human breast cancers, seven adjacent normal breast samples and three normal breast organoid preparations was extracted. Bisulfite sequencing showed that the entire region of the VDR promoter remained largely unmethylated in the three normal organoid samples, whereas the breast tissue adjacent to breast cancer showed low levels of aberrant methylation, i.e., with ˜5-15% of CpGs methylated (see lower portion of FIG. 3). In contrast, CpGs were found highly methylated in the primary breast tumors at essentially the same residues as were observed in the breast cancer cell lines. More than 40-65% of the CpG dinucleotides were methylated in cancer tissues, albeit with some inter-sample variability, particularly around the −760 bp region of the VDR promoter.

### Example 3

### Quantitative MSP Assays Confirm VDR Promoter Hypermethylation in Breast Cancer Tissue

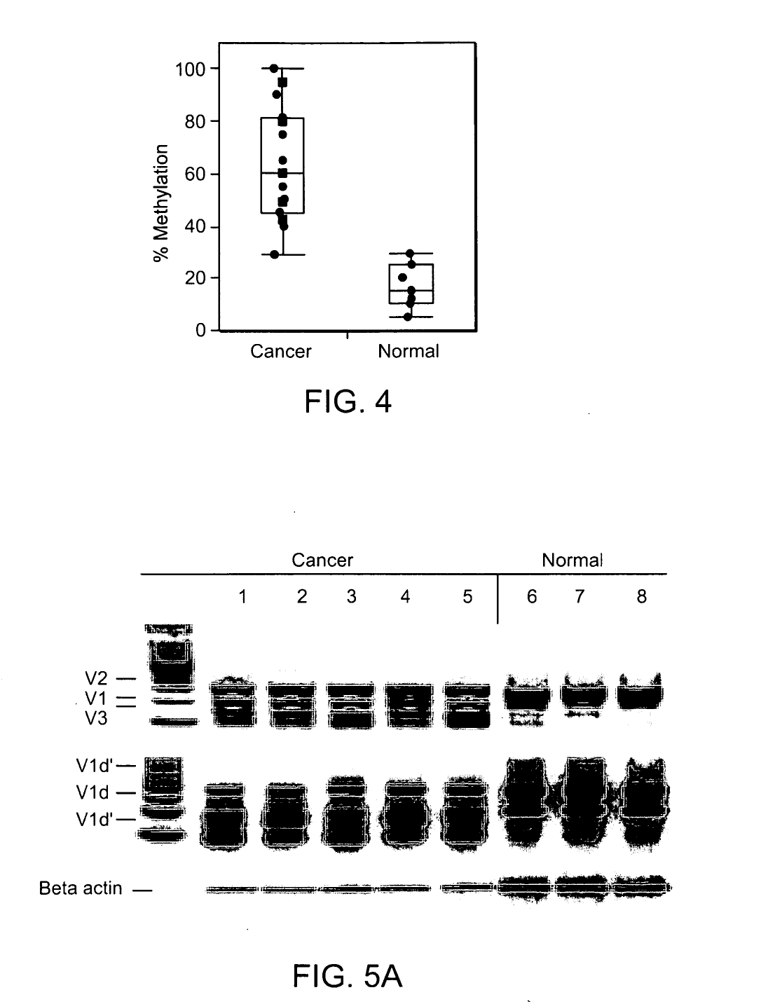

To independently validate the bisulfite sequencing results, MSP primers were designed and a group of primary breast cancer tissues (n=15; 10 fresh frozen and 5 FFPE tissues) and adjacent normal breast tissues (n=7 fresh frozen samples), were assayed using methylated (M) and unmethylated (UN) DNA-specific primers directed to the 5′ VDR promoter. As shown in FIG. 4, breast cancer samples showed significantly greater CpG hypermethylation (average 65%) than normal breast tissue (average 15%) (Wilcoxon rank sum test: p<0.0002). These results confirmed that the VDR gene promoter is robustly hypermethylated in breast cancer, compared to normal cells, and support the proposed mechanism of silencing expression of VDR through gene promoter hypermethylation and of sensitizing cells to Calcitriol through demethylation of VDR and subsequent VDR re-expression.

### Example 4

### Alternatively Spliced VDR Transcripts Predominate in Primary Breast Cancers

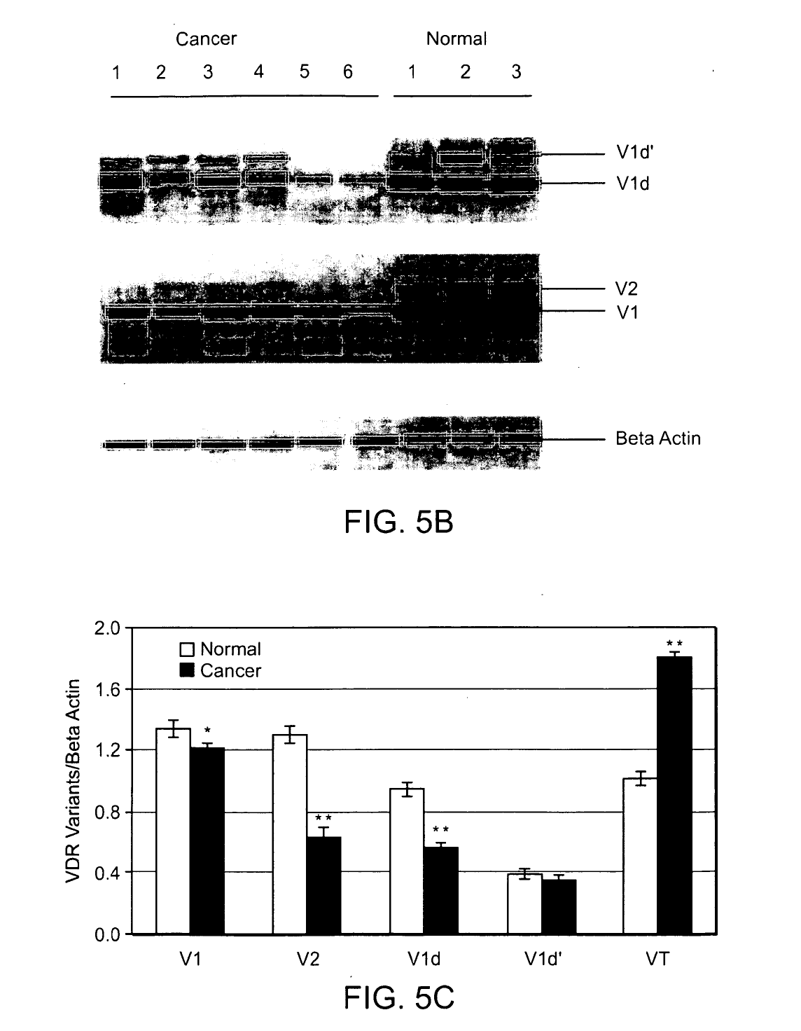

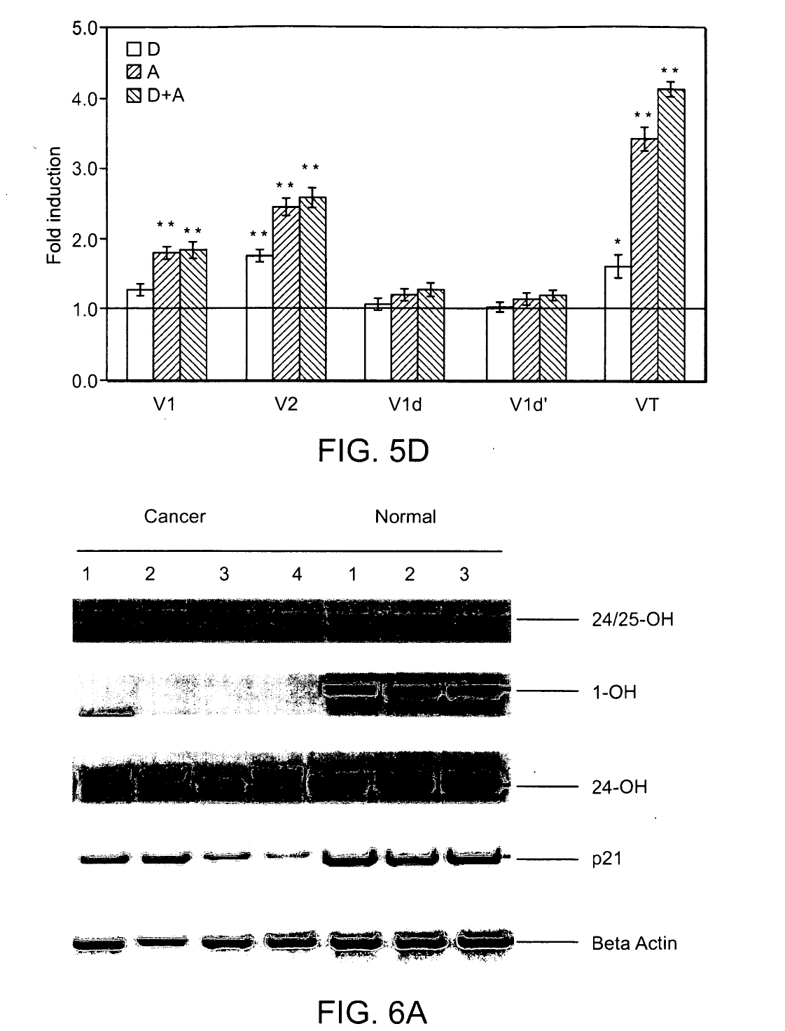

Interestingly, when primary breast cancer tissues were compared to normal breast tissues, significantly higher levels of total VDR transcript were observed in primary cancer (FIG. 5). Because of the apparent inconsistency between these data and that generated from breast cancer cell lines (FIG. 2), a comparative analysis of alternative VDR transcripts in primary cancer tissues was performed. Use of secondary VDR promoters and translational start sites as well as alternative splicing can generate a variety of VDR transcripts and proteins (Crofts et al., Proc Natl Acad Sci USA 1998; 95:10529-34; Esteban et al., Biochem Biophys Res Commun 2005; 334:9-15; Gardiner et al., J Steroid Biochem Mol Biol 2004; 89-90:233-8; Sunn et al., Mol Endocrinol 2001; 15:1599-609). Therefore, the electrophoretic patterns generated using PCR primer sets designed to detect several 5′ splice variants of VDR were examined.

FIG. 1 illustrates a number of splice variant patterns in relation to the exon structure of VDR. These are generally referred to using the nomenclature of Crofts et al (supra). VDR primer set V1 detects three variants that respond to the classic VDR promoter just upstream of exon 1a, and which encode the standard 427aa VDR peptide, also known as VDRA. Primer set V1d detects three additional variants that use alternative splicing of the 1d exon to encode proteins with N-terminally extended domains, VDRB1 (477aa) and VDRB2 (450aa). The V3 and V1d″ splice variants have not been shown to be translated into protein (Sunn, supra), as indicated by the asterisk.

Based on these patterns, a series of PCR experiments using the V1 and V1d primer sets specific for Exon 1 and Exon 2 transcript variants were performed. As shown in FIG. 5A, the levels and patterns of splice variants of VDR were markedly different in primary cancer compared to normal breast epithelial tissue, with the cancer tissue showing extensive heterogeneity and variability, particularly in the shorter variants that are barely detectable in normal tissue. The V1, V2, V1d, and V1d′ RT-PCR products were sequence-verified, and measured by quantitative imaging of PCR reactions performed at lower amplification cycle numbers (28 cycles), in order to ensure amplification in the linear range of the reactions (shown in FIG. 5B). As shown in the bar graphs in FIG. 5C, the results from six primary breast cancer samples and three normal breast organoids indicate that all major full length 5′ splice variants encompassed by these four primers sets were present at lower levels in primary breast cancers when compared to normal breast tissue. Thus, these splice variants alone cannot account for the elevation in total VT transcript observed in patient cancer samples compared to normal samples. A significant fraction of the VDR transcripts found in breast cancer appears to be truncated and generated from more downstream regions.

### Example 5

### VDR Promoter Methylation May Influence V1 and V2 VDR Variant Expression

To investigate if VDR methylation affects expression of the active forms of VDR, the VDR variant expression patterns in the same mRNA preparations obtained for the in vitro experiments shown in FIG. 2 were analyzed. As shown in FIG. 5D, which summarizes the results in the five cell lines, Calcitriol alone had a mild effect, while levels of V1 and V2 were significantly increased after AZA treatment in all the breast cell lines. V1d and V1d′ variants were less responsive to demethylation. The addition of Calcitriol to AZA showed little additive effect on the variants examined. Results indicate that at least the V1 and V2-specific promoter is partially regulated by DNA methylation.

### Example 6

### VDR Response Genes are Expressed at Low Levels in Breast Cancer Tissues

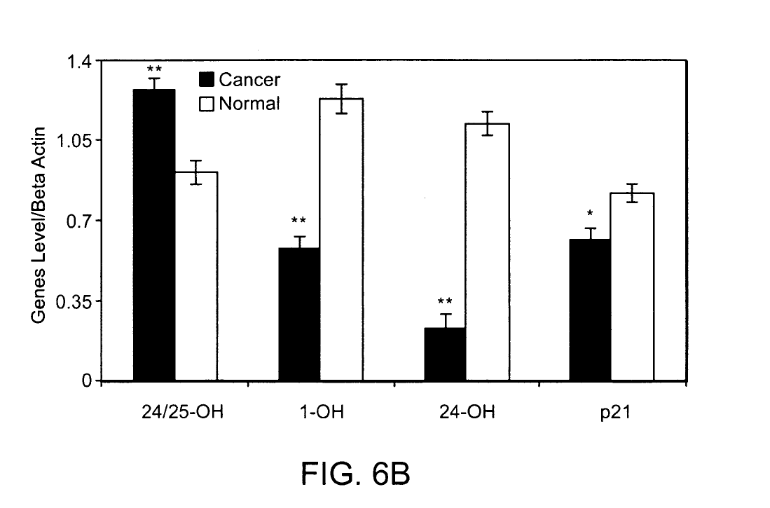

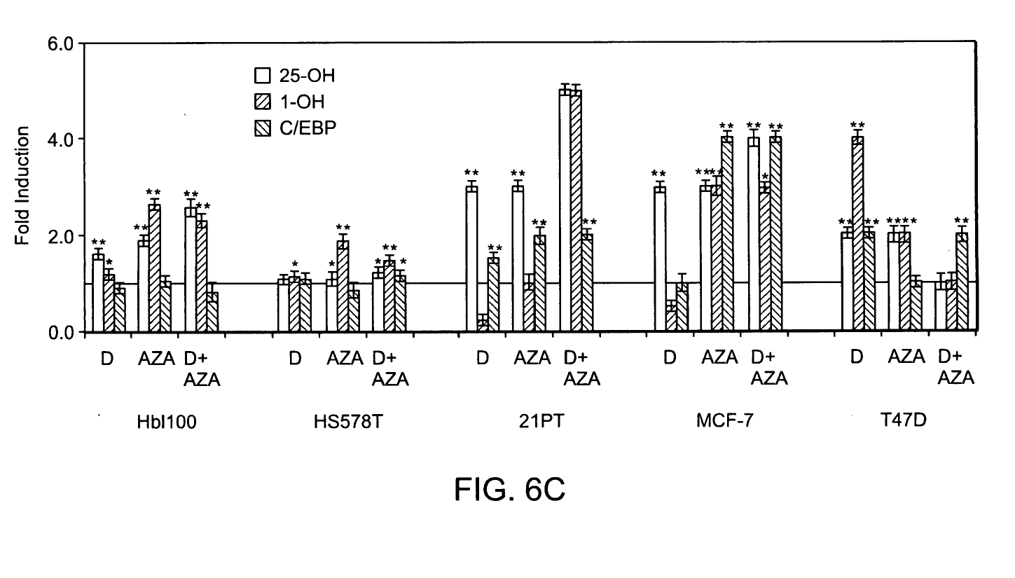

The status of four VDRE-containing VDR responsive genes in breast cancer tissue was interrogated by measuring their expression levels. Several cytochrome p450 hydroxylases are known to contain VDREs, including CYP27B1 (the Calcitriolactivating 1-hydroxylase, 1-OH), CYP24A1 and CYP3A4 (Calcitriol-inactivating 24-OH and 24/25-OH hydroxylases, respectively) that are part of a negative feedback loop, and p21, which functions as tumor suppressor. As illustrated in FIG. 6, the expression of all VDR downstream genes examined, with the exception of CYP3A4, are higher in normal breast tissue than in cancer tissue. These data suggest that some alternative transcripts expressed in primary breast cancer tissues may be functionally distinct from normal full length VDR transcripts, in that they may not fully activate VDR-responsive genes.

These findings demonstrate that the VDR promoter is hypermethylated in breast cancer, and provide evidence that demethylation of the receptor in breast cancer cell lines results in re-expression of the VDR transcripts. Correspondingly, while treatment of breast cancer cells with VTD alone is ineffective in inhibiting cell growth, concurrent treatment with AZA is associated with increasing VDR expression and results in highly effective inhibition of breast cancer cell growth in vitro.

Similar epigenetic suppression of VDR mRNA expression has been demonstrated in placental carcinoma cell lines, which also showed VDR mRNA re-expression after AZA treatment (Pospechova et al., Mol Cell Endocrinol 2009; 299:178-87). Initial proliferation assays confirmed the Calcitriol insensitivity in breast cancer cells (see FIG. 2A), and restoration of sensitivity after AZA treatment, with an additive response to Calcitriol when both AZA and Calcitriol were used together. Changes in VDR transcript levels mirrored these results (see FIG. 2B). These results were validated in clinical samples, where clear hypermethylation of a CpG island in the VDR promoter region in breast cancer tissue was shown, which was reversed by treating cells with AZA. Therefore, the VDR promoter is hypermethylated in breast cancer, and demethylation of the receptor results in re-expression of the VDR transcripts. Thus, much of the unresponsiveness of the VTD pathway in breast cancer is likely due to epigenetic silencing of VDR transcription, and this may be reversible by pharmacological intervention using demethylating agents.

Numerous conflicting or inconclusive studies have made it difficult to arrive at definitive conclusions regarding the mechanism of Calcitriol insensitivity and the biological impact of this mechanism in cancer. Data on VDR expression levels in cancer have been particularly inconsistent. Quantitative RT-PCR of VDR has shown that its expression was modestly down-regulated in endometrial cancer cells₃₀ and in human colon and lung tumor samples, compared to their normal counterparts, while it was strongly overexpressed in ovarian cancer tissue (Cancer Chemother Pharmacol 2006; 57:234-40). A closer analysis of the specific transcript regions amplified may help reconcile reports describing conflicting levels of VDR expression in breast cancer tissue compared to adjacent normal or independent control tissue. These results suggested that a significant proportion of transcripts detected in breast cancer tissue by standard real-time RT-PCR may be N-terminally truncated and may not yield functional peptides, while full-length transcripts are relatively depressed, compared to normal breast epithelial tissue. Very little is known about the possible roles in breast cancer of the various isoforms of VDR. Initial reports describing several VDR promoters and multiple transcripts with tissue-specific abundance variation were followed by more detailed biochemical analyses documenting functional differences between the VDRA, VDRB1 and VDRB2 isoforms (Esteban Biochem Biophys Res Commun 2005; 334:9-15; Gardiner et al., J Steroid Biochem Mol Biol 2004; 89-90:233-8). There are currently few data, however, on how this regulatory complexity affects breast cancer epidemiology or pathogenesis, let alone what role the potentially untranslated, N-terminally truncated variants detected in our breast cancer samples might have. A detailed analysis of VDR transcripts and their functional significance is beyond the scope of this report, but RT-PCR data on VDR expression levels, which strongly depended on the specific amplicons used, suggest that any analysis failing to take into account the heterogeneity of transcripts in breast cancer tissue could be misleading. In this study, demethylation treatment of breast cancer cell lines with AZA induced the re-expression of active variant transcripts of VDR, V1 and V2. These results suggest a correlation between VDR methylation and active transcript variant expression, at least for the promoter controlling the VDRA isoforms.

Further complicating the role of VDR in breast carcinogenesis, Calcitriol metabolism is controlled by a complex interplay of genetic, nutritional and environmental factors. For example, the dietary intake of Vitamin D only contributes about 10% to Calcitriol synthesis, while the UV-initiated cutaneous conversion of 7-dehydrocholesterol to Vitamin D accounts for about 90% (Norman, Am J Clin Nutr 1998; 67:1108-10). Vitamin D then undergoes a two-step activation process. The initial 25-hydroxylation is performed predominantly in the liver, and the resulting 250H-VTD is bound to the D-binding transport protein (DBP), constitutes a reservoir, and is the most commonly measured Vitamin D metabolite. 250H-VTD levels vary considerably depending on access to sunlight and skin pigmentation. The production of active Calcitriol by the rate-limiting 1α-hydroxylase CYP27B1 is under tight control in the kidney. It has recently become apparent, however, that CYP27B1 is expressed in a wide range of cell types, including breast epithelial cells, where it is subject to post-transcriptional 35 negative feedback inhibition (Schwartz et al., Carcinogenesis 2004; 25:1015-26; Townsend et al., Clin Cancer Res 2005; 11:3579-86; Lechner et al., Mol Cell Endocrinol 2007; 263:55-64). Antiproliferative and differentiating effects of CYP27B1 have been detected in many different tissues (van den Bemd et al., Curr Drug Targets 2002; 3:85-94).

Similarly, there is a positive feedback loop with CYP24A1, which is induced by ligand activated VDR. CYP24A1 hydroxylates Calcitriol at the 24-position, the first step in its degradation pathway. Investigation of the expression levels of VDRE-containing, Calcitriol metabolizing p450 hydroxylases showed that, consistent with a low Calcitriol pathway activity, the rate limiting activating 1α-hydroxylase and the main catabolic 24-hydroxylase mRNAs are underexpressed in breast cancer tissues (FIG. 6). Data from clinical specimens have been contradictory, however, with reports of up- or downregulation of the opposing 1- or 24-hydroxylases. Some of these findings could be attributed to expression of splice-variants encoding non-functional proteins, as reported in several cancers, including breast cancer. Interestingly, consistently increased levels of CYP3A4, which has a strong 24-hydroxylase activity and is involved in the metabolism of several steroid hormones and in xenobiotic metabolism. CYP3A4 polymorphisms have been identified as potential risk factors for predisposition to breast and prostate cancer, and may have pharmacogenetic implications in the tumor response to several chemotherapeutic agents as well (Suman et al., Cancer Biomark 2009; 5:33-40). In the context of a downregulated Calcitriol pathway in breast cancer, CYP3A4's expression may be less influenced by its VDRE than by triggers related to its role in steroid hormone and xenobiotic metabolism, and its Calcitriol catabolic actions may further depress already low levels of Calcitriol in breast tissue.

Decreased expression of p21 (CDKN1A) has been reported in breast cancer and in ovarian cancer. p21 contains three VDREs, and is known to be regulated by Calcitriol-induced cyclical chromatin looping (Saramaki et al., J Biol Chem 2009; 284:8073-82). In agreement with these reports, decreased expression of the p21 tumor suppressor gene was found in breast cancer samples, as would be expected in the context of an inactive Calcitriol pathway (see FIG. 6).

In summary, this report provides further evidence for the importance of the Calcitriol/VDR axis in breast cancer, and suggests that potentially reversible epigenetic silencing may be at the center of its inactivation.

### Cell Lines and In Vitro Pharmacological Assays

The 21PT human breast cancer cell line was derived from a primary tumor and was propagated as described (Band et al., Cancer Res 1990; 50:7351-7). Human breast cell lines were obtained from American Type Culture Collection (Rockville, Md.) and propagated as described 17. For pharmacological assays, 1.0×10⁶ cells were seeded in 25 cm² tissue culture flasks. After 24 hours, the culture media were changed and cells were treated with vehicle alone, or 1.5 μM 1_{—}α 25(OH)₂ D3 (Calcitriol, Calcitriol), 7.5 μM 5′ deoxy-azacytidine (AZA) (Sigma, St. Louis, Mo.) or both (1.5 μM Calcitriol and 7.5 μM AZA) for 96 hours.

### MTT Assay of Inhibition of Cellular Proliferation

Cell viability was measured using the Cell Titer 96 AQ-One Solution Cell Proliferation Assay kit from Promega Corporation (Madison, Wis.). Formazan absorbance was read at 490 nm in a 96-well plate reader.

### Breast Epithelial Tissue Organoid Isolation

Normal breast tissue organoids were prepared from reduction mammoplasty specimens of women without breast abnormalities as previously described (Bergstraesser et al., Cancer Res 1993; 53:2644-54). All tissue samples were obtained with the approval of the Johns Hopkins Institutional Review Board.

### Tissue Collection

Primary breast cancer tissues (Invasive ductal carcinomas, pT2-3NxMx) were obtained after surgical resection at the Johns Hopkins Hospital (Baltimore Md.), anonymized, and stored frozen at −80° C. or fixed in 10% buffered formalin and embedded in paraffin (FFPE). Samples containing >50% tumor cells were processed for molecular studies.

### DNA and RNA Extraction

RNA and DNA were purified from cell cultures and tissue samples by organic extraction using the Trizol Reagent (Invitrogen Inc., Carlsbad, Calif.). Total cellular RNA and DNA were quantified by UV absorption at 260 nm using a Nanodrop spectrophotometer (NanoDrop Technologies Inc, Wilmington, Del.).

### Bisulfite Sequencing Analysis of CpG Methylation

DNA from cell lines or tissue (1 μg) was treated with sodium bisulfite as previously described (Herman et al., Proc Natl Acad Sci USA 1996; 93:9821-6. Three sets of bisulfite sequencing primers P1, P2 and P3 were designed (see Table 1), encompassing the region from 790 bp upstream to 380 bp downstream of the VDR transcription start site. Primers were tagged with 21 bp of the M13 universal primer at the 5′-end to facilitate subsequent sequencing reactions.

PCR products were separated electrophoretically and isolated using a PCR purification kit (Qiagen, Valencia, Calif.). DNA was sequenced using the M13 Reverse Primer with an Applied Biosystems automated fluorescent sequencer according to the manufacturer's instructions. Percent DNA methylation was determined using the ratio of cytidine to thymidine traces at CpG dinucleotides.

Table 1 describes primers useful in the methods of the invention. Nucleotide positions throughout this disclosure are defined relative to NT_{—}029419.12. In particular, nucleotide positions 10442909-10441739 within the Genbank sequence are numbered 1->1170, herein.

TABLE 1

| | | GRCh37 NT_029419.12 [symbol] c10442909 <- | |
|---|---|---|---|
| VDR [Gene ID 7421] | | 10.441739 = 1 -> 1170 | Nucleotide positions |
| Bisulfite-Sequencing | | Primer Sequences: | {NT_229419.12) |
| Primers: | | | |
| P1F | | M13- ATT TAT AAT TTT AGG TTT TAG GAG GTA GTT | 20-49 |
| P1R | | M13- TCA CCC CCA CCT AAA CTA ACC AAA CCA | 405-431 |
| P2F | | M13- ATT TTA TTT TAA TTT GTG GGA TTA GGT TGA | 251-280 |
| P2R | | M13- CCA ATC CTC TCT TAC CAA AAA CTC C | 686-710 |
| P3F | | M13- GTT TAT AGG GTG GTT GAT TTT AAG TTA AGA | 640-669 |
| P3R | | M13- AAA CAA ATA CTT CTT ATT ACC CAA ATA CTA | 1136-1165 |
| Methylation-Specific | | | {NT_029419.12) |
| PCR Primers: | | | |
| MF | | TTT TTT TTA CGT CGA TGT TAC G | 58-79 |
| MR | | ATG GGA AAT TTC GGG TTT CG | 318-337 |
| UNF | | TTT TTT TTA TGT TGA TGT TAT GG | 58-80 |
| UNR | | AAT GGG AAA TTT TGG GTT TTG | 319-338 |
| Total VDR RT-PCR | | | |
| Primers: | | | |
| VF | (Exon 3) | ACT TTG ACC GGA ACG TGC CC | 204-223 (NM_00376.2) |
| VR | (Exon 4) | CAT GCC GAT GTC CAC ACA | 410-427 (NM_00376.2) |
| [symbol] ′ Variant RT-PCR | | | |
| Printers: | | | |
| V1F | (Exon 1a) | CAA AAG GCG GCA GCG GAG C | 9-27 (NM_00376.2) |
| V1R = V1dR | (Exon 2) | CCG CCA TTG CCT CCA TCC C | 158-176 (NM_00376.2) |
| V1dF | (Exon 1d) | GGC ATG GAG TGG AGG AAT AA | 371-399 (BC_060832.1) |

Table 1 Legend:
Primers used. M13- indicates M13 universal sequencing primer.
For the genomic probes, numbers indicate nucleotide positions relative to the-strand of the specified Genbank sequence from the GRCh37 reference primary assembly. [symbol]
indicates data missing or illegible when filed

Quantitative MSP (qMSP) Assay

The VDR P1 region was pre-amplified from bisulfite treated DNA and the products were isolated as above. QMSP was then performed using methylated (M) and unmethylated (UN) DNA-specific primers with the Qiagen SYBR green PCR Kit (Qiagen, Valencia, Calif.) in an ABI PRISM 7900HT instrument. Percent methylation was calculated as the ratio of inverse Ct values of methylated DNA/methylated plus unmethylated DNA.

### Quantitative Real-Time RT-PCR (QRT-PCR)

Reverse transcription reactions were performed as previously described (Crofts et al., Proc Natl Acad Sci USA 1998; 95:10529-34) using random hexamer primers. PCR was then performed for 40 cycles using gene-specific primers. To determine total VDR levels, pre-mixed “Assay on Demand” primer-probe sets for VDR (Hs01045843_m1) and GAPDH (Hs99999905_m1) and the GeneAmp® Fast PCR Master Mix were purchased from Applied Biosystems (Foster City, Calif.) and cDNA was amplified following the manufacturer's instructions in the ABI PRISM 7900HT instrument. This VDR primer probe set amplifies a region between Exon 7 and 8 and does not distinguish between the VDR splice variants that were investigated in this report.

### VDR Splice-Variant Specific Primer Design

The V1 primers, spanning exon 1a and exon 2, were designed to recognize variant V1 and V2 transcripts (see FIG. 1). The V1d primers, spanning exon 1d and the junction of exon 1c and exon 2, were designed to recognize the transcript variants v1d, V1d′ and v1d″ (FIG. 1). In order to detect all of these variants, the nonspecific V6 primers, spanning exon 3 and exon 4, were used.

### Quantitation of RT-PCR Products

Published primer sets and reaction conditions were used for RT-PCR assays of cytochrome hydroxylases (24/25-OH, CYP3A4 21, 1-OH, CYP27B1 22, and 24-OH, CYP24A1 23), and p21 (Zheng et al., Cell Death Differ 2006; 13:1960-7). Predetermined linear ranges of the amplification reactions, i.e. 25 to 28 cycles for VDR and its splice variants, and 30 to 35 cycles for the Calcitriol hydroxylases and p21, were chosen for semiquantitative analyses of transcript levels. The PCR products were separated on 2% agarose gels, scanned in a Gel Doc Imager (BioRad, Philadelphia, Pa.), and quantified using Image Quant software (BioRad, Philadelphia, Pa.).

### Cloning and Sequencing of RT-PCR Products

PCR products identified after RT-PCR using the V1 and V1d primers were excised from the agarose gel, isolated using the QIAEX II gel extraction kit (Qiagen, Valencia, Calif.), and cloned using the TOPO TA cloning Kit (Invitrogen Inc., Carlsbad, Calif.). Expected plasmid inserts were verified by EcoR1 digestion and sequenced using the M13 reverse primer.

### Statistical Analysis

Experiments were performed in triplicate to achieve consistent results. For each experiment, data were expressed as means [symbol] standard deviation except where stated otherwise. Significance was determined using the two-tailed Student t-Test or the Wilcoxon rank sum test.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for characterizing a breast carcinoma in a biologic sample, the method comprising quantifying the promoter methylation of the vitamin D receptor in the sample, wherein an increased quantity of promoter methylation relative to a reference indicates that the breast carcinoma is vitamin D-resistant.

2. A method for detecting a breast carcinoma in a biologic sample, the method comprising quantifying the promoter methylation of the vitamin D receptor in the sample, wherein an increased quantity of promoter methylation relative to a reference indicates the presence of a neoplasia in the sample.

3. The method of claim 1 or 2, wherein promoter methylation is quantified using bisulfite sequencing.

4. The method of claim 3, wherein sequencing is performed between nucleic acids 790 bp upstream and 380 bp downstream of the VDR transcription start site.

5. The method of claim 3, wherein the primers interrogate areas of high methylation between about −760 and −450.

6. The method of claims 1-4, further comprising measuring the expression of a VDR downstream gene selected from the group consisting of CYP27B1, CYP24A1 CYP3A4 and p21.

7. The method of claim 5, wherein the method detects an increase in one or more of CYP27B1, CYP24A1 and p21 in cancer tissue relative to normal tissue.

8. The method of claim 5, wherein the method detects a decrease in CYP3A4 in cancer tissue relative to normal tissue.

9. The method of claims 1-4, wherein the reference is the level of methylation present at the promoter in a control sample.

10. The method of claim 9, wherein the control sample is derived from a healthy subject.

11. The method of claims 1-4, wherein the promoter methylation is quantified using quantitative methylation-specific PCR (QMSP).

12. The method of any one of claims 1-4, wherein the biologic sample is a patient sample.

13. A method of selecting a treatment for a subject diagnosed as having breast carcinoma, the method comprising:
(a) quantifying the level of vitamin D receptor promoter methylation in a biologic sample from the subject relative to a reference, wherein the level of promoter methylation is indicative of a treatment; and
(b) selecting a treatment.

14. The method of claim 13, wherein the method detects a hypermethylated region.

15. The method of claim 14, wherein detection of a hypermethylated region identifies the breast carcinoma as vitamin D-resistant.

16. The method of claim 15, wherein the treatment selected for a vitamin D-resistant carcinoma is Calcitriol and a demethylating agent or HDAC inhibitor.

17. A method of monitoring a subject diagnosed as having breast carcinoma, the method comprising quantifying the level of vitamin D receptor promoter methylation in a sample derived from the subject, wherein an altered level of promoter methylation relative to the level of methylation in a reference indicates an altered severity of carinoma in the subject.

18. The method of claim 17, wherein the reference is the level of methylation present in a sample previously obtained from the subject.

19. The method of claim 17, wherein the reference is a baseline level of methylation present in a sample from the subject obtained prior to therapy.

20. The method of claim 17, wherein the reference is the level of methylation present in a normal patient sample.

21. The method of claim 17, wherein a reduced level of promoter methylation indicates a reduced severity of neoplasia.

22. The method of claim 17, wherein detection of no alteration in the level of promoter methylation indicates no reduction in the severity of the neoplasia.

23. A method of identifying a subject as having a propensity to develop a breast carcinoma, the method comprising obtaining a breast tissue sample from the subject, quantifying the level of vitamin D receptor promoter methylation in the sample, wherein an altered level of promoter methylation relative to the level of methylation in a reference identifies the subject as having a propensity to develop a breast carcinoma.

24. The method of claim 23, wherein the tissue sample comprises a precancerous lesion.

25. The method of claim 24, wherein the precancerous lesion is selected from the group consisting of simple hyperplasia, atypical hyperplasia, and breast carcinoma in.

26. The method of claim 24, wherein the tissue sample is obtained in as a core biopsy or fine needle aspirant.

27. A method of treating or preventing vitamin D-resistant breast carcinoma in a subject, the method comprising administering to the subject an effective amount of Calcitriol and a demethylating agent or an HDAC inhibitor.

28. The method of claim 26, wherein the histone deacetylase inhibitor is selected from the group consisting of Scriptaid, varinostat, APHA Compound 8, Apicidin, sodium butyrate, (−)-Depudecin, HMBA, valproic acid, Sirtinol, trichostatin A, and salts or analogs thereof.

29. The method of claim 26, wherein the demethylating agent sensitizes cells to Calcitriol.

30. A kit for the analysis of promoter methylation, the kit comprising at least one primer capable of distinguishing between methylated and unmethylated promoter, and directions for using the primer for the analysis of promoter methylation.

31. A kit for the analysis of promoter methylation, the kit comprising primers useful for bisulfite sequencing, and directions for using the primers for the analysis of vitamin D receptor promoter methylation.

32. A kit for the analysis of vitamin D receptor promoter methylation, the kit comprising at least one primer capable of distinguishing between methylated and unmethylated vitamin D receptor promoter, and directions for using the primer for the analysis of promoter methylation.

33. The kit of claim 31 or 32, further comprising a pair of primers a reference gene.

34. A collection of primer sets, each of the primer sets comprising at least two primers that bind to vitamin D receptor promoter, wherein at least one of the primers is capable of distinguishing between methylated and unmethylated vitamin D receptor promoter.

35. A collection of primer sets, wherein each of the primer sets comprises at least two primers capable of amplifying a sequence comprising a methylated region of the vitamin D receptor promoter.

36. The collection of claim 34 or 35, wherein the collection comprises a sequence shown in Table 1.

37. A pharmaceutical composition comprising an effective amount of calcitriol and a HDAC inhibitor or demethylating agent in a pharmaceutically acceptable excipient.

38. The composition of claim 36, wherein the demethylating agent is 5-azacyitidine and the histone deacetylase inhibitor is selected from the group consisting of Scriptaid, varinostat (e.g., SAHA), APHA Compound 8, Apicidin, sodium butyrate, (−)-Depudecin, HMBA, valproic acid, Sirtinol, trichostatin A, and salts or analogs thereof.

39. A method for characterizing a breast carcinoma, the method comprising detecting the expression of one or more vitamin D receptor variants in the sample, wherein detection of an increased number of such variants relative to a reference indicates that the breast carcinoma is vitamin D-resistant.

40. A method for detecting a breast carcinoma in a biologic sample, the method comprising detecting the expression of a vitamin D receptor or variants thereof in the sample, wherein detection of an increased level of total vitamin D receptor transcripts or variants thereof relative to a reference indicates the presence of a carcinoma in the sample.

41. The method of claim 39 or 40, wherein the method detects 5′ splice variants.

42. A collection of primers that amplifies a sequence encoding a vitamin D receptor transcript or variant thereof.

43. The collection of claim 42, wherein the collection comprises a sequence of Table 1.


SEQUENCE LISTING (16 sequences)

SEQ ID NO: 1 - DNA, 1171 bp, Homo sapiens
tggtgagttttccgcagccatccacaattccaggtctcaggaggtagtctttatcttttcccctcacgccgatgccacggggcggggggggaaggcggaactcgggaccagggaccagggaagctgagactcagctcccttgggtgagattcgcgacaggccgggaacgtggtcagccgcggtcgctgccaaggtgatatcgggtgggagcaatgacgcaactccggtttccacttcggccccccgggatattttaccctaatctgtgggatcaggctgagcttcctggcgttctgcagcagtaacaggttggcgagcggagcccgggatttcccattcgtgcggagctagccgccggtgccagtcggcaggcgccccccagcgtcccgcggacgacgaagtcctggcctggtcagcccaggtgggggtgacgcacctggctcaggcgtccgcagcaggctgggtagaaccacggcaggaagggtggggggctgcatccccgattaacacaggctgaagcgggtatccgcacctataatcatcgacaactctgtcccacagagggcagaagcgtgccttgccctatggacgacggtcgatgaaaatttcacgagttagagtatctaaggctacagcgtggcctatagggtggttgattccaagtcaagatggttgcagcgccaacggagctcctggcaagagaggactggacctgtgggcggggcggaggggcggggcggggccggggcggggcctgaccgagaggcggggccaggtgctgggctgtctctgcttgtcaaaaggcggcagcggagccgtgtgcgccgggagcgcggaacagcttgtccacccgccggccggaccaggtgcgaacccgggagcagcgggaaagggggtctcaggatagggactcggggtcggggcgtctgggataccggggcctgagcgcccggctgcgagcattagagtctaagtctcagcggtaaacttggctactgaggtccgggctgtcgtgccatgaggctgggacactaaggggcactgaggtttgagaaagctgaagttcgtgccaggctggcgaggggagcagcgacatcctcggcgcttaggagaaatgctccgctaacacagtgcttagcacttgggcaacaagaagtatttgtttccttct

SEQ ID NO: 2 - DNA, 30 bp, Artificial Sequence
  Description of Artificial Sequence Synthetic primer
atttataattttaggttttaggaggtagtt

SEQ ID NO: 3 - DNA, 27 bp, Artificial Sequence
  Description of Artificial Sequence Synthetic primer
tcacccccacctaaactaaccaaacca

SEQ ID NO: 4 - DNA, 30 bp, Artificial Sequence
  Description of Artificial Sequence Synthetic primer
attttattttaatttgtgggattaggttga

SEQ ID NO: 5 - DNA, 25 bp, Artificial Sequence
  Description of Artificial Sequence Synthetic primer
ccaatcctctcttaccaaaaactcc

SEQ ID NO: 6 - DNA, 30 bp, Artificial Sequence
  Description of Artificial Sequence Synthetic primer
gtttatagggtggttgattttaagttaaga

SEQ ID NO: 7 - DNA, 30 bp, Artificial Sequence
  Description of Artificial Sequence Synthetic primer
aaacaaatacttcttattacccaaatacta

SEQ ID NO: 8 - DNA, 22 bp, Artificial Sequence
  Description of Artificial Sequence Synthetic primer
ttttttttacgtcgatgttacg

SEQ ID NO: 9 - DNA, 20 bp, Artificial Sequence
  Description of Artificial Sequence Synthetic primer
atgggaaatttcgggtttcg

SEQ ID NO: 10 - DNA, 23 bp, Artificial Sequence
  Description of Artificial Sequence Synthetic primer
ttttttttatgttgatgttatgg

SEQ ID NO: 11 - DNA, 21 bp, Artificial Sequence
  Description of Artificial Sequence Synthetic primer
aatgggaaattttgggttttg

SEQ ID NO: 12 - DNA, 20 bp, Artificial Sequence
  Description of Artificial Sequence Synthetic primer
actttgaccggaacgtgccc

SEQ ID NO: 13 - DNA, 18 bp, Artificial Sequence
  Description of Artificial Sequence Synthetic primer
catgccgatgtccacaca

SEQ ID NO: 14 - DNA, 19 bp, Artificial Sequence
  Description of Artificial Sequence Synthetic primer
caaaaggcggcagcggagc

SEQ ID NO: 15 - DNA, 19 bp, Artificial Sequence
  Description of Artificial Sequence Synthetic primer
ccgccattgcctccatccc

SEQ ID NO: 16 - DNA, 20 bp, Artificial Sequence
  Description of Artificial Sequence Synthetic primer
ggcatggagtggaggaataa